(19) United States
(12) Patent Application Publication
(10) Pub. No.: US 2010/0129848 A1
(43) Pub. Date: May 27, 2010

(54) Rapid Mixing Device for Subsecond Analysis of Cell Surface Kinetics in Flow Cytometry

(75) Inventors: James Bogert, Flagstaff, AZ (US); Richard Posner, Flagstaff, AZ (US); Larry A. Sklar, Albuquerque, NM (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Arizona Board of Regents , a body Corporate acting for and on behalf of Northern Arizona Univ., Scottsdale, AZ (US)

(21) Appl. No.: 12/375,931

(22) PCT Filed: Aug. 28, 2007
(86) PCT No.: PCT/US2007/077005

§ 371 Date: Oct. 28, 2009

Related U.S. Application Data
(60) Provisional application No. 60/841,065, filed on Aug. 30, 2006.

(51) Int. Cl. C12Q 1/02; C12M 1/24

(52) U.S. Cl. 435/29; 435/288.1

(57) ABSTRACT

The present invention provides improved flow cytometry tubes containing ports to allow rapid addition and mixing of reagents with sample during flow cytometry. The present invention further provides rapid mixing cytometry devices and method for their use in rapid reagent mixing during flow cytometry.

### CROSS REFERENCE

This application claims priority from U.S. Provisional Patent Application Ser. No. 60/841,065 filed Aug. 30, 2006, the disclosure of which is incorporated by reference herein in its entirety.

### BACKGROUND OF THE INVENTION

In flow cytometry, cells are passed one at a time through a flow cell, which is adapted for sensing or detecting impedance changes, light scatter or some other characteristic of the cell. Some flow cytometry instruments are equipped with detectors for measuring emissions from fluorescent tags that may be associated with the cells, while other detectors measure scatter intensity or pulse duration. Data about cells that pass through the flow cell can be plotted according to the measured property. (U.S. Pat. No. 6,794,152)

When studying the kinetics of cell surface reactions and the impact of added test samples, such as ligands and/or ligand competitors (“reagents”), one needs to gather data as quickly as possible after reagent mixing. Two strategies currently exist for mixing cells with ligands and/or ligand competitors just prior to measurement. The simplest approach involves using a regular cytometry tube and manually mixing the two reagents while the tube is detached from the cytometer. Using this method, it is impossible to determine the gap between the mixing of the cells and ligand(s) and measurement of the desired property. Estimates of the time gap range between five and ten seconds. With a gap this large, many reactions are at or near equilibrium before the first measurement is taken, as many cell surface reactions have half lives of less than 5 seconds.

Another approach for rapidly mixing reagents prior to measurement is use of a Rapid Mix Flow Cytometer (RMFC). A RMFC consists of 3 computer controlled syringe drives, a series of sample loops and a delay line connected to two miniature solenoid valves and a specially modified cytometer. Two of the syringes (S1 and S2) are loaded with a sample to be mixed. These syringes push the samples through respective sample loops to a mixing tree where the samples meet and enter the delay line. Once the samples have been mixed, they are in the delay line and need to be pushed into the cytometer. To accomplish this, the solenoid valves, which had been directing fluid to a waste container during the mixing, are switched to a direct flow into the modified cytometer. The third syringe is then used to push the mixed samples through the delay line and into the cytometer for data collection. After every experiment, the delay line must be flushed with a buffer solution to avoid contamination of the subsequent experiment.

Given the drawbacks in current methods for reagent mixing in flow cytometry, improved devices and methods for reagent mixing are needed in the art.

### SUMMARY OF THE INVENTION

In a first aspect, the present invention provides improved flow cytometry tubes, the improvement comprising the inclusion of one or more ports in the flow cytometry tube. In various preferred embodiments, the improved flow cytometry tube comprises two or more ports in the flow cytometry tube; the location of the one or more ports are at or near the level of the liquid to be placed in the flow cytometer tube during flow cytometry; the improved flow cytometry tube further comprises one or more port tubes, wherein each port tube is adapted to fit through an individual port on the flow cytometry tube; and the improved flow cytometry tube further comprises a gasket to seal an interface between the port and the port tubing.

In a second aspect, the present invention provides rapid mixing cytometry devices, comprising:

(a) the improved flow cytometry tube of the first aspect of the invention;

(b) one or more port tubes comprising a distal end, a proximal end, and an outer surface, wherein the distal end of the one or more port tubes passes through the one or more ports and is in fluid communication with the improved flow cytometry tube, and wherein the proximal end of each port tube is in fluid communication with a reservoir;

(c) a gasket between the port and the outer surface of the port tube; and

(d) a flow cytometer in fluid communication with the improved flow cytometry tube. In various preferred embodiments, movement of fluid from each reservoir to the improved flow cytometry tube is controlled by one or more pumps; and the one or more pumps are computer controlled.

In a third aspect, the present invention provides methods for reagent mixing in a flow cytometry tube comprising:

(a) providing the rapid mixing cytometry device of the second aspect of the invention, wherein a sample is located within the flow cytometry tube; and

(b) delivering one or more reagents to the improved flow cytometry tube through the one or more port tubes, to permit mixing of the one or more reagents within the improved flow cytometry tube.

### BRIEF DESCRIPTION OF THE FIGURES

FIG. 1 is a non-limiting example of the improved flow cytometry tube of the present invention.

FIG. 2 is another non-limiting example of the improved flow cytometry tube of the present invention.

FIG. 3 is a non-limiting example of a rapid mix cytometry device according to the present invention.

### DETAILED DESCRIPTION OF THE INVENTION

In a first aspect, the present invention provides an improved flow cytometry tube, the improvement comprising one or more ports in the flow cytometry tube. Improved cytometry tubes according to this first aspect of the invention are also referred to herein as “rapid mix cytometry tubes” (RMCT). These tubes can be used to provide rapid mixing of reagents into samples present in the flow cytometer tube, thus overcoming the drawbacks of prior art methods and devices. Details of the use of the improved flow cytometer tubes of this first aspect of the invention in rapid mixing are provided below.

As used herein, a “flow cytometry tube” is any tube specifically designed to work with a flow cytometer. Current embodiments of flow cytometer tubes have specific dimensions at the mouth of the tube (12 mm) and the length of the tube (75 mm) These two dimensions are currently what make a tube compatible with a flow cytometer. The mouth of the tube must make a seal with the flow cytometer because fluid is driven into the flow cytometer via pressure in the cytometer tube. The length of the cytometer tube is important because there is an arm on the cytometer that holds the tube tightly against the cytometer to maintain the seal. However, it will be clear to those of skill in the art that the improved flow cytometry tubes of the present invention can be adapted to different dimensions of the mouth and tube length if future embodiments of flow cytometers are compatible with such other tube dimensions.

As used herein, a “port” is an opening through the cytometry tube for the passage of molecule, compound, test sample, etc., whether gas, fluid, or particulate, that one might want to add to the contents of a flow cytometer tube. Any number of ports can be added to the cytometry tube, so long as each port can be separately accessed for reagent addition. In one embodiment, the improved flow cytometer tube comprises 1-10 ports; thus, in various embodiments, the improved flow cytometer tube can comprise 1, 2, 3, 4, 5, 6, 7, 8, 9, or 10 ports. The size of the ports is not critical to the invention so long as each port can be separately accessed for reagent addition. The only limit on how small a port can be is that any port tubing to be used (see below) must be able to pass though the port. For example, if a 1/16^{th} inch OD port tubing is to be used, the port must be sized to accommodate it, and should be approximately 1.6 mm diameter or larger. In various other embodiments, the one or more ports range in diameter between 1 mm and 2 mm. The diameter of the ports in a given improved flow cytometry tube can vary; thus, in a non-limiting example of an improved flow cytometry tube with 3 ports, one could have a diameter of 1 mm, another could have a diameter of 1.5 mm, and the third could have a diameter of 2.0 mm. Those of skill in the art will understand that many such variations are possible.

The specific location of the one or more ports on the improved flow cytometry tube is also not critical. Preliminary results have demonstrated that the mixing results are similarly good regardless of where the one or more ports are located. In a preferred embodiment, the location of the one or more ports are at or near the level of the liquid to be placed in the flow cytometer tube during flow cytometry, to ensure good mixing of the two fluids. However, the one or more ports can also be placed above or below the fluid line, because so much turbulence is created when more liquid is introduced at an extremely fast rate. It would be clear to one of ordinary skill in the art that the pressure produced by the cytometer would not be great enough to cause fluid to leak out of the cytometry tube when placement of the one or more ports is below the fluid line. In one embodiment, the one or more ports are located between 0.5 cm and 2.0 cm from the closed end of the flow cytometry tube. Furthermore, when more than one port is used, the ports do not have to be the same distance from the bottom/top of the tube (ie: do not have to be in the same plane of the improved flow cytometer tube).

In a further embodiment of the first aspect, the improved flow cytometry tube further comprises one or more port tubes, wherein each port tube is adapted to fit through an individual port on the flow cytometry tube. Once inserted, the port tube thus provides fluid communication between the improved flow cytometry tube and a reservoir to which the port tube is also in fluid communication. In one embodiment, the port tube is connected to the improved flow cytometry tube, wherein a distal end of the port tube is inside the improved flow cytometry tube, and a proximal end is outside the tube of the improved flow cytometry tube. In this embodiment, the improved flow cytometer tube can be provided connected to the one or more port tubes, or separate, wherein the one or more port tubes are connected to the improved flow cytometer by a user prior to use.

The port tubing can be any such tubing suitable for use with the improved flow cytometry tube. Thus, any tubing that can hold the pressure of a cytometer can be used. In one embodiment, an outside diameter (O.D.) of the port tubing is 1/16^{th} of an inch, and the inside diameter (I.D.) is any suitable ID as determined by the needs of a given experiment. Any suitable material can be used for the port tubing, including but not limited to Teflon tubing. In a further embodiment, the port tubing can comprise metal port tubing that is built into the improved cytometer tube. In a further embodiment, the metal port tubing is adapted to be connected to a distal end of a flexible port tube, such as a Teflon tube.

The driving pressure needed to move the sample through the flow cell is provided by the cytometer. Since the inside of the improved flow cytometry tube is pressurized for this purpose, a means for sealing the ports is preferred. Thus, in a further embodiment, the improved flow cytometry tube further comprises a gasket to seal the interface between the port and the port tubing. As used herein, a “gasket” is any substance or material that prevents air or liquid from escaping through the port around the outside of the port tubing while under pressure. Such gaskets can be made of any suitable material, including but are not limited to gasket paper, rubber, silicone, metal, felt, fiberglass, plastic, and glass from the improved flow cytometry tube forming a seal against the port tubing (such as the metal port tubing embodiment).

The gasket can be of any size useful for serving the purpose of preventing air or liquid from escaping around the outside of the port tubing when connected through the port to the improved flow cytometry tube. The gasket can be self-sealing, or it may utilize further compounds for sealing, such as epoxy resins.

The methods and devices of the invention allow for very short dead time between mixing the reagents and measuring, for example, their fluorescence and extended time for data collection. Furthermore, the flow cytometry tube of the invention does not need to be disconnected from the cytometer for mixing.

In another aspect, the present invention provides a rapid mixing cytometry device, comprising:

(a) the improved flow cytometry tube disclosed above;

(b) one or more port tubes comprising a distal end, a proximal end, and an outer surface, wherein the distal end of the one or more port tubes passes through the one or more ports and is in fluid communication with the improved flow cytometry tube, and wherein the proximal end of each port tube is in fluid communication with a reservoir;

(c) a gasket between the port and the outer surface of the port tube; and

(d) a flow cytometer in fluid communication with the improved flow cytometry tube.

As used herein, “in fluid communication” means that fluid can pass between the components recited as being in fluid communication.

As used herein a “reservoir” is a source of reagents to be added to the improved flow cytometry tube. Any container that can hold liquid that can be extracted from it and forcefully pushed into the cytometer tube can be used as the reservoir. The reservoir can be in direct fluid communication with the proximal end of the port tubing, or may be indirectly connected through one or more intermediate connections. It will be understood that each port tube can be in fluid communication with the same reservoir, different reservoirs, or combinations thereof. It is further preferred that movement of the reagents from reservoir to port tubing is controlled by one or more pumps. One non-limiting example of such a reservoir is a syringe in which reagents to be added to the flow cytometry tube are placed. In this embodiment, it is preferred that reagent movement from the syringe reservoir into the port tubing is controlled by one or more syringe pumps. Syringe pumps (such as those manufactured by Cavro) provide very predictable and consistent mixing times and volumes, and thus are especially useful for rapid mix cytometry, although the methods of the invention can be carried out using a syringe that is controlled by hand.

In a further embodiment, each port tube is in fluid communication with a different reservoir and reservoir pump. In a non-limiting example of such an embodiment, a small amount of Reagent A is placed in the improved flow cytometry tube and a large amount of Reagent B is placed in a syringe driven by a syringe pump. When the experiment is run, a small amount of Reagent B is inserted into the improved flow cytometry tube and the cytometer reads the AB mixture. For the next trial, the improved flow cytometry tube (that now contains both Reagent A and Reagent B) is disconnected from the syringe and the flow cytometer, and a fresh improved flow cytometry tube with only Reagent A is connected to both. Trials can be run in this fashion until the syringe (that contains Reagent B) is emptied.

In a further preferred embodiment, the one or more reservoir pumps are computer controlled. Such computer-controlled reservoir pumps (including, but not limited to syringe pumps), are well known in the art.

In another aspect, the present invention provides methods for reagent mixing in a flow cytometry tube comprising:

(a) providing the rapid mixing cytometry device according to any of the embodiments described above, wherein a sample is located within the flow cytometry tube;

(b) delivering one or more reagents to the improved flow cytometry tube through the one or more port tubes, to permit mixing of the one or more reagents within the improved flow cytometry tube.

In a preferred embodiment, the methods utilize one or more computer-controlled pumps (such as syringe drives) to rapidly infuse one or more reagents into the improved flow cytometry tube, also referred to as the rapid mix cytometry tube (RMCT). This allows two or more different liquid substances to be mixed together without interrupting flow into the cytometer. The rapid mix cytometry tube can comprise a normal sized cytometry tube with one or more ports that allows one to insert multiple reagents into the tube as measurements are being made, as described above.

### Example 1

### Improved Cytometry Tubing

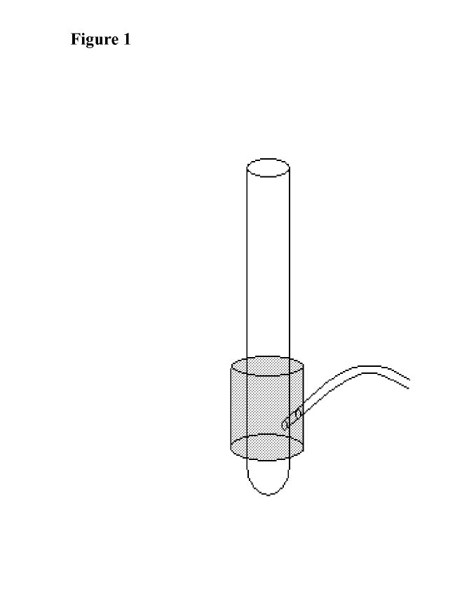

In the example shown in FIG. 1, a small hole is made near the closed end of a cytometry tube. The hole is then covered with a silicon sheath. The port tubing is inserted through the silicon sheath into the port. Epoxy resin is used to seal this area around the tubing. When the end of the tubing is connected to a syringe, the rapid mix cytometry tube (RMCT) can hold pressure, and the contents of the syringe can be added at any time. In some embodiments, it is preferred that the smallest possible total volume is added to the RMCT through the one or more port tubes.

### Example 2

### Temperature Control Flow Cytometry

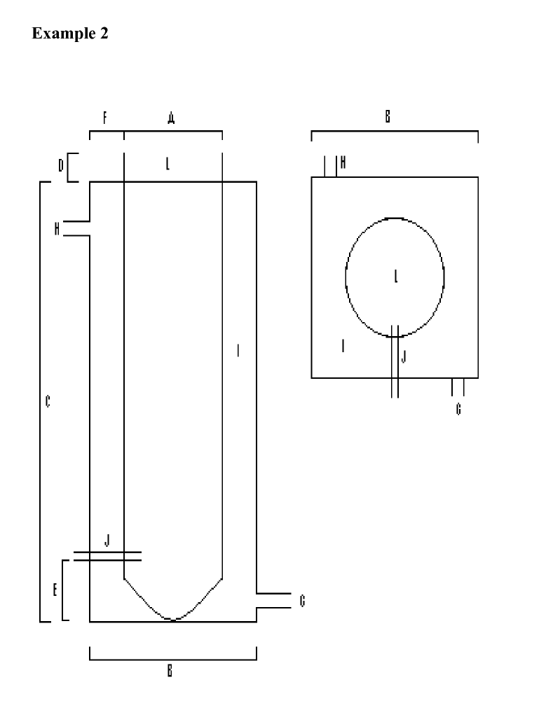

In the example shown in FIG. 2, a device according to the invention is adapted for control of the temperature at which the experiment is run. In this example the rapid mix cytometry tube is made of glass. The glass cytometry tube (L) is contained within a reservoir for water (I), which is used to control the temperature at which the experiment is run. This concept is similar to condensation tubes used in organic chemistry labs. This does not change the functionality of the mixing but, since some cytometry experiments must be done at body temperature, this allows for temperature control and provides added functionality. The glass cytometry tube contains metal port tubing (J). The metal port tubing is built into the glass and the reservoir contains both an input (G) and output (H) for water. In one non-limiting example, the width of the mouth of the cytometry tube which makes a seal with the cytometer (A) is 12 mm; the distance that the cytometry tube extends above the water reservoir (D) is 15 mm; the width of the water reservoir (B) is 24 mm, the height of the water reservoir (C) is 60 mm, and the distance between the cytometry tube and the walls of the water reservoir (F) is 6 mm in order to accommodate current standard cytometry tubes; and the distance of the metal port tubing from the bottom of the cytometry tube is 10-20 mm (E) and.

### Example 3

### Rapid Mixing Flow Cytometry

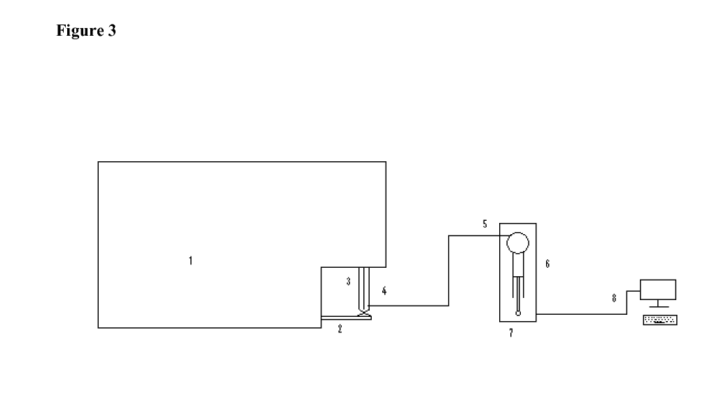

In the example shown in FIG. 3, the improved, rapid mixing, flow cytometer tube (3) containing the sample is attached to both the cytometer (1) via the cytometer arm (2) which holds the cytometry tube tight against the cytometer to produce a seal, and the distal end of the port tubing (4). The proximal end of the port tubing (5) is connected to a syringe (6) holding the substance to be mixed with the sample. A computer controlled syringe driver (7), which moves the syringe, is connected (8) to and controlled by a computer.

We claim:

1. An improved flow cytometry tube, the improvement comprising one or more ports in the flow cytometry tube.

2. The improved flow cytometry tube of claim 1 comprising two or more ports in the flow cytometry tube.

3. The improved flow cytometry tube of claim 1 further comprising one or more port tubes, wherein each port tube is adapted to fit through an individual port on the flow cytometry tube.

4. The improved flow cytometry tube of claim 3 further comprising a gasket to seal an interface between the port and the port tubing.

5. A rapid mixing cytometry device, comprising:
(a) the improved flow cytometry tube of claim 1;
(b) one or more port tubes comprising a distal end, a proximal end, and an outer surface, wherein the distal end of the one or more port tubes passes through the one or more ports and is in fluid communication with the improved flow cytometry tube, and wherein the proximal end of each port tube is in fluid communication with a reservoir;
(c) a gasket between the port and the outer surface of the port tube; and
(d) a flow cytometer in fluid communication with the improved flow cytometry tube.

6. The rapid mixing cytometry device of claim 5 wherein movement of fluid from each reservoir to the improved flow cytometry tube is controlled by one or more pumps.

7. The rapid mixing cytometry device of claim 5 wherein the one or more pumps are computer controlled.

8. A method for reagent mixing in a flow cytometry tube comprising:
(a) providing the rapid mixing cytometry device of claim 5, wherein a sample is located within the flow cytometry tube;
(b) delivering one or more reagents to the improved flow cytometry tube through the one or more port tubes, to permit mixing of the one or more reagents within the improved flow cytometry tube.
